SEQUENCE LISTING (12 sequences)

SEQ ID NO: 1 - protein, 556 aa, synthetic construct [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

(19) United States
(12) Patent Application Publication
(10) Pub. No.: US 2025/0382366 A1
(43) Pub. Date: Dec. 18, 2025

(54) METHODS OF PRODUCING ANTI-CD19 ANTIBODIES

(71) Applicant: Zenas BioPharma, Inc., Waltham, MA (US)

(72) Inventors: Mei Shao, Waltham, MA (US); Weiyi Liu, Waltham, MA (US)

(21) Appl. No.: 19/242,372

(22) Filed: Jun. 18, 2025

Related U.S. Application Data
(60) Provisional application No. 63/661,248, filed on Jun. 18, 2024.

(51) Int. Cl. C07K 16/28

(52) U.S. Cl. CPC C07K 16/2803; C07K 2317/14; C07K 2317/24; C07K 2317/31; C07K 2317/41

(57) ABSTRACT

The present invention provides compositions and methods of producing antibodies or antigen-binding fragment that specifically bind human CD19.

### CROSS-REFERENCE TO RELATED APPLICATIONS

This application claims priority to, and the benefit of, U.S. provisional application No. 63/661,248, filed on Jun. 18, 2024, the content of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Jun. 16, 2025, is named “ZEN-018US1_SequenceListing” and is 13,128 bytes.

### BACKGROUND

The design and development of biologic therapeutics such as antibodies provides new challenges in manufacturing, such as those relating to the yield and purity of these novel therapeutics at commercial scale. For example, standard processes may be unable to achieve the desired product profile while being both efficient and economical. Therefore, there remains a need for inventing new, highly optimized manufacturing processes capable of producing antibodies with the desired product quality attributes.

### SUMMARY

Among other things, the present disclosure provides improved methods and compositions for production of anti-CD19 antibodies including those described herein. Methods of manufacturing described herein can provide anti-CD19 antibodies (e.g., those described herein) with favorable characteristics for therapeutic use, such as desirable N-glycan profiles and/or reduced presence of low molecular weight species impurities (e.g., antibody fragments). For example, methods according to the present invention can successfully produce anti-CD19 antibodies (e.g., those described herein such as obexelimab) with a high sialyation and low mannose-5, including at large scale. Moreover, methods described herein can also reduce the presence of low molecular weight species. Accordingly, the present invention can provide highly efficient methods for manufacturing anti-CD19 antibodies with therapeutically beneficial features.

In one aspect, the present invention provides a composition comprising an anti-CD19 antibody comprising a light chain variable region comprising a lCDR1 defined by SEQ ID NO: 1, a lCDR2 defined by SEQ ID NO: 2, and a lCDR3 defined by SEQ ID NO: 3, and a heavy chain variable region comprising a hCDR1 defined by SEQ ID NO: 4, a hCDR2 defined by SEQ ID NO: 5, and a hCDR3 defined by SEQ ID NO: 6, wherein the anti-CD19 antibody comprises an Fc region comprising S267E and L328F substitutions, and wherein the anti-CD19 antibody comprises an N-glycan profile comprising ≥20% sialylated glycans.

In some embodiments, the N-glycan profile of the antibody comprises ≥25% sialylated glycans. In some embodiments, the N-glycan profile of the antibody comprises ≥26%, 28%, or 30% sialylated glycans.

In some embodiments, the N-glycan profile of the antibody comprises ≤5%, 4%, 3%, 2% or 1% mannose-5 glycans. In some embodiments, the N-glycan profile of the antibody comprises ≤1%, 0.9%, 0.8%, 0.7%, 0.6%, or 0.5% mannose-5 glycans.

In one aspect, the present invention provides a composition comprising an anti-CD19 antibody comprising a light chain variable region comprising a lCDR1 defined by SEQ ID NO: 1, a lCDR2 defined by SEQ ID NO: 2, and a lCDR3 defined by SEQ ID NO: 3, and a heavy chain variable region comprising a hCDR1 defined by SEQ ID NO: 4, a hCDR2 defined by SEQ ID NO: 5, and a hCDR3 defined by SEQ ID NO: 6, wherein the anti-CD19 antibody comprises an Fc region comprising S267E and L328F substitutions, and wherein the anti-CD19 antibody comprises an N-glycan profile comprising ≤1% mannose-5 glycans.

In some embodiments, the N-glycan profile of the antibody comprises ≥20%, 21%, 22%, 23%, 24%, 25%, 26%, 27%, 28%, 29%, or 30% sialylated glycans. In some embodiments, the N-glycan profile of the antibody comprises ≥25% sialylated glycans.

In some embodiments, the anti-CD19 antibody comprises a light chain variable region that comprises SEQ ID NO: 7 and a heavy chain variable region that comprises SEQ ID NO: 8.

In some embodiments, the anti-CD19 antibody comprises a light chain comprising SEQ ID NO: 9 and a heavy chain comprising SEQ ID NO: 10.

In one aspect, the invention features a composition comprising an anti-CD19 antibody comprising a light chain comprising SEQ ID NO: 9 and a heavy chain comprising SEQ ID NO: 10; wherein the anti-CD19 antibody comprises an N-glycan profile comprising ≥20%, 25%, 26%, 28%, or 30% sialylated glycans.

In one aspect, the invention features a composition comprising an anti-CD19 antibody comprising a light chain comprising SEQ ID NO: 9 and a heavy chain comprising SEQ ID NO: 10; wherein the anti-CD19 antibody comprises an N-glycan profile comprising ≤5%, 4%, 3%, 2%, 1%, 0.9%, 0.8%, 0.7%, 0.6%, or 0.5% mannose-5 glycans.

In some embodiments, the composition comprises ≤100 ng/mg host cell protein (HCP). In some embodiments, the composition comprises ≤75 ng/mg host cell protein (HCP). In some embodiments, the composition is a liquid composition.

In some embodiments, the anti-CD19 antibody is present at a concentration ranging from 100-150 mg/ml. In some embodiments, the anti-CD19 antibody is present at a concentration ranging from 113 to 138 mg/mL.

In some embodiments, the liquid composition has a pH ranging from 5 to 6. In some embodiments, the pH is 5.5.

In some embodiments, the liquid composition has an osmolality of 265 to 441 mOsm/kg.

In some embodiments, the liquid composition has a viscosity less than 10 cP at 25° C.

In some embodiments, the composition comprises a main charge variant that constitutes ≥28% of charge variants by icIEF-Desialylation.

In some embodiments, the composition comprises a main monomer peak that constitutes ≥95% of the total antibody measured by SE-UPLC Purity.

In some embodiments, the composition comprises high molecular weight species that constitute ≤5.0% of the total antibody measured by SE-UPLC Purity.

In some embodiments, the composition comprises low molecular weight species that constitute ≤5.0% of the total antibody measured by SE-UPLC Purity.

In some embodiments, the composition comprises ≥90% intact antibody as measured by non-reduced CE-SDS.

In one aspect, the invention provides a liquid composition comprising an anti-CD19 antibody at a concentration ranging from 113-138 mg/ml, wherein the anti-CD19 antibody comprises a light chain variable region comprising a lCDR1 defined by SEQ ID NO: 1, a lCDR2 defined by SEQ ID NO: 2, and a lCDR3 defined by SEQ ID NO: 3, and a heavy chain variable region comprising a hCDR1 defined by SEQ ID NO: 4, a hCDR2 defined by SEQ ID NO: 5, and a hCDR3 defined by SEQ ID NO: 6, and wherein the anti-CD19 antibody comprises an Fc region comprising S267E and L328F substitutions, wherein the composition comprises a main charge variant that constitutes ≥28% of charge variants by icIEF-Desialylation.

In one aspect, the invention provides a liquid composition comprising an anti-CD19 antibody at a concentration ranging from 113-138 mg/ml, wherein the anti-CD19 antibody comprises a light chain variable region comprising a lCDR1 defined by SEQ ID NO: 1, a lCDR2 defined by SEQ ID NO: 2, and a lCDR3 defined by SEQ ID NO: 3, and a heavy chain variable region comprising a hCDR1 defined by SEQ ID NO: 4, a hCDR2 defined by SEQ ID NO: 5, and a hCDR3 defined by SEQ ID NO: 6, and wherein the anti-CD19 antibody comprises an Fc region comprising S267E and L328F substitutions, wherein ≥95% of the anti-CD19 antibody is present as monomer measured by SE-UPLC.

In one aspect, the invention provides a comprising an anti-CD19 antibody, wherein the anti-CD19 antibody comprises a light chain comprising SEQ ID NO: 9 and a heavy chain comprising SEQ ID NO: 10; wherein the composition comprises a main charge variant that constitutes ≥28% of charge variants by icIEF-Desialylation.

In one aspect, the invention provides a liquid composition comprising an anti-CD19 antibody at a concentration ranging from 113-138 mg/ml, wherein the anti-CD19 antibody comprises a light chain comprising SEQ ID NO: 9 and a heavy chain comprising SEQ ID NO: 10; wherein ≥95% of the anti-CD19 antibody is present as monomer measured by SE-UPLC.

In one aspect, the invention provides a liquid composition comprising an anti-CD19 antibody at a concentration ranging from 113-138 mg/ml, wherein the anti-CD19 antibody comprises a light chain comprising SEQ ID NO: 9 and a heavy chain comprising SEQ ID NO: 10; wherein ≤5% of the anti-CD19 antibody is present as low molecular weight species measured by SE-UPLC.

In some embodiments, ≤5% of the anti-CD19 antibody is present as high molecular weight species measured by SE-UPLC.

In some embodiments, ≤5% of the anti-CD19 antibody is present as low molecular weight species measured by SE-UPLC.

In one aspect, the invention provides a method of manufacturing an anti-CD19 antibody, comprising culturing mammalian cells engineered to express the anti-CD19 antibody in a fed-batch production process under conditions such that cell viability ≥95% is maintained during the fed-batch production process.

In some embodiments, cell viability ≥96%, ≥97%, ≥98% or ≥99% is maintained during the fed-batch production process.

In some embodiments, the conditions comprise one or more of the following steps: feeding cells with glutamine at day 2 of the fed-batch production process; feeding cells with galactose at day 2 of the fed-batch production process; feeding cells with asparagine at day 2 of the fed-batch production process; feeding cells with serine at day 2 of the fed-batch production process; feeding cells with tyrosine at day 2 of the fed-batch production process; feeding cells with cystine at day 2 of the fed-batch production process; feeding cells with galactose between day 6 and day 11 of the fed-batch production process; feeding cells with cystine at day 5 of the fed-batch production process; and providing glucose between day 6 and day 11 of the fed-batch production process. In some embodiments, the method comprises feeding tyrosine at day 2 of the fed-batch process. In some embodiments, the method comprises feeding cystine at day 2 of the fed-batch process. In some embodiments, the method comprises feeding cystine at day 5 of the fed-batch process.

In some embodiments, the conditions comprise a step of controlling pCO2 between 80 to 130 mmHg during the fed batch production process. In some embodiments, the step of controlling pCO2 between 80 to 130 mmHg occurs from day 4 to day 10 of the fed batch process.

In some embodiments, the conditions comprise controlling lactate dehydrogenase (LDH)<1500 U/L during the fed batch production process.

In some embodiments, the conditions comprise culturing the mammalian cells at a target temperature of 37° C. during the fed batch production process.

In some embodiments, the conditions comprise a step of seeding the mammalian cells at a cell density of 0.3×10⁶ to 0.5×10⁶ cells/mL.

In some embodiments, the conditions comprise shifting the mammalian cells from a target temperature of 37° C. to a target temperature of 34° C. when the fed batch production process exceeds a cell density of 5.0×10⁶ cells/mL. In some embodiments, the step of shifting the mammalian cells from a target temperature of 37° C. to a target temperature of 34° C. when the fed batch production process exceeds a cell density of 5.0×10⁶ cells/mL target temperature occurs between day 3 and day 5 of the fed batch production process.

In some embodiments, the mammalian cells are CHO cells.

In some embodiments, the CHO cells are engineered to express an anti-CD19 antibody comprising a light chain variable region comprising a lCDR1 defined by SEQ ID NO: 1, a lCDR2 defined by SEQ ID NO: 2, and a lCDR3 defined by SEQ ID NO: 3; and a heavy chain variable region comprising a hCDR1 defined by SEQ ID NO: 4, a hCDR2 defined by SEQ ID NO: 5, and a hCDR3 defined by SEQ ID NO: 6, and wherein the anti-CD19 antibody comprises an Fc region comprising S267E and L328F substitutions. In some embodiments, the light chain variable region comprises SEQ ID NO: 7 and the heavy chain variable region comprises SEQ ID NO: 8. In some embodiments, the anti-CD19 antibody comprises a light chain comprising SEQ ID NO: 9 and a heavy chain comprising SEQ ID NO: 10.

In some embodiments, the CHO cells engineered to express the anti-CD19 antibody express the anti-CD19 antibody at more than one integration site.

In some embodiments, the fed batch production process is performed at a scale of at least 10 L, at least 50 L, at least 100 L, at least 200 L, at least 500 L, at least 1000 L, at least 1500 L, or at least 2000 L.

In one aspect, the invention provides a method of manufacturing an anti-CD19 antibody, comprising culturing mammalian cells engineered to express the anti-CD19 antibody in a fed-batch production process under conditions such that cell viability ≥95% is maintained during the fed-batch production process, wherein the conditions comprise a step of controlling pCO2 between 80 to 130 mmHg during the fed batch production process, and wherein the anti-CD19 antibody comprises a light chain comprising SEQ ID NO: 9 and a heavy chain comprising SEQ ID NO: 10.

In one aspect, the invention provides a method of manufacturing an anti-CD19 antibody, comprising: culturing mammalian cells engineered to express the anti-CD19 antibody in a fed-batch production process under conditions such that cell viability ≥95% is maintained during the fed-batch production process; wherein the anti-CD19 antibody comprises a light chain comprising SEQ ID NO: 9 and a heavy chain comprising SEQ ID NO: 10; and wherein the conditions comprise one or more of the following steps: feeding cells with glutamine at day 2 of the fed-batch production process; feeding cells with galactose at day 2 of the fed-batch production process; feeding cells with asparagine at day 2 of the fed-batch production process; feeding cells with serine at day 2 of the fed-batch production process; feeding cells with tyrosine at day 2 of the fed-batch production process; feeding cells with cystine at day 2 of the fed-batch production process; feeding cells with galactose between day 6 and day 11 of the fed-batch production process; feeding cells with cystine at day 5 of the fed-batch production process; and providing glucose between day 6 and day 11 of the fed-batch production process.

In one aspect, the invention provides a method of manufacturing an anti-CD19 antibody, comprising: culturing mammalian cells engineered to express the anti-CD19 antibody in a fed-batch production process under conditions such that cell viability ≥95% is maintained during the fed-batch production process; wherein the conditions comprise controlling lactate dehydrogenase (LDH)<1500 U/L during the fed batch production process; and wherein the anti-CD19 antibody comprises a light chain comprising SEQ ID NO: 9 and a heavy chain comprising SEQ ID NO: 10.

In one aspect, the invention provides a method of manufacturing an anti-CD19 antibody, comprising: culturing mammalian cells engineered to express the anti-CD19 antibody in a fed-batch production process under conditions such that cell viability ≥95% is maintained during the fed-batch production process; wherein the conditions comprise a step of seeding the mammalian cells at a cell density of 0.3×10⁶ to 0.5×10⁶ cells/mL and shifting the mammalian cells from a target temperature of 37° C. to a target temperature of 34° C. when the fed batch production process exceeds a cell density of 5.0×10⁶ cells/mL; and wherein the anti-CD19 antibody comprises a light chain comprising SEQ ID NO: 9 and a heavy chain comprising SEQ ID NO: 10.

In one aspect, the invention provides an anti-CD19 antibody manufactured according to a method described herein. In another aspect, the invention features a composition comprising an anti-CD19 antibody manufactured according to a method described herein. In some embodiments, a composition is any composition described herein.

### BRIEF DESCRIPTION OF DRAWING

The figures described below, that together make up the Drawings, are for illustration purposes only, not for limitation.

FIG. 1A depicts the relationship between the integral of viable cell density (IVCD) and the partial pressure of CO₂ (pCO2), demonstrating reduced cell growth between day 4 and day 10 of the production process when cells are cultured under conditions of increasing partial pressure of CO₂.

FIG. 1B depicts the relationship between IVCD and lactic acid dehydrogenase (LDH), demonstrating an increased amount of LDH in the cell culture as IVCD increases.

FIG. 2A depicts the relationship between the percentage of antibody fragments and LDH, demonstrating an increased abundance of antibody fragments as cell culture LDH increases.

FIG. 2B depicts the relationship between the percentage of antibody fragments and cell viability as measured by trypan blue exclusion, demonstrating an increased abundance of antibody fragments as cell culture viability decreases.

FIG. 3 depicts a chromatogram of the N-glycan profile of obexelimab under different harvest criteria, demonstrating an increase in highly sialyated glycans when high cell viability is maintained throughout the entire duration of the production process.

### DETAILED DESCRIPTION

### Definitions

Described herein are several definitions. Such definitions are meant to encompass grammatical equivalents.

Antibody: The term “antibody” as used herein means a protein consisting of one or more polypeptides substantially encoded by all or part of the recognized immunoglobulin genes. The recognized immunoglobulin genes, for example in humans, include the kappa (K), lambda (1), and heavy chain genetic loci, which together comprise the myriad variable region genes, and the constant region genes mu (u), delta (d), gamma (γ), sigma(s), and alpha (a) which encode the IgM, IgD, IgG (lgG1, lgG2, lgG3, and lgG4), IgE, and IgA (lgA1 and lgA2) isotypes respectively. Antibody herein is meant to include full length antibodies and antibody fragments, and may refer to a natural antibody from any organism, an engineered antibody, or an antibody generated recombinantly for experimental, therapeutic, or other purposes.

Baseline: The term “baseline” is defined as values of a parameter prior to commencement of treatment with a therapeutic. In some embodiments “baseline” is an initial measurement of a condition that is taken at an early time point and used for comparison over time to look for changes. In some embodiments, the baseline is time “zero”, before the participants in the study receive an experimental agent or intervention, or negative control; drug safety and efficacy may be determined by monitoring changes in baseline values.

CD32b⁺ cell or FcγRIIb⁺ cell: The terms “CD32b⁺ cell” or “FcγRIIb⁺ cell” as used herein means any cell or cell type that expresses CD32b (FcγRIIb). CD32b+ cells include but are not limited to B cells, plasma cells, dendritic cells, macrophages, neutrophils, mast cells, basophils, or eosinophils.

CDC or complement dependent cytotoxicity: The terms “CDC” or “complement dependent cytotoxicity” as used herein is meant the reaction wherein one or more complement protein components recognize bound antibody on a target cell and subsequently cause lysis of the target cell.

Effector Function: The term “effector function” as used herein is means a biochemical event that results from the interaction of an antibody Fc region with an Fc receptor or ligand. Effector functions include FcγR-mediated effector functions such as ADCC and ADCP, and complement-mediated effector functions such as CDC. Further, effector functions include FcγRIIb-mediated effector functions, such as inhibitory functions (e.g., downregulating, reducing, inhibiting etc., B cell responses, e.g., a humoral immune response).

Effector Cell: The term “effector cell” as used herein means a cell of the immune system that expresses one or more Fc and/or complement receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gd T cells, and may be from any organism including but not limited to humans, mice, rats, rabbits, and monkeys.

Fc or Fc region: The terms “Fc” or “Fc region,” as used herein means the polypeptide comprising the constant region of an antibody excluding the first constant region immunoglobulin domain and in some cases, part of the hinge. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” as used herein means any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V1 58 and F158) and FcγRIIIb (including allotypes FcγRIIIb-NA1 and FcγRIIIb-NA2) (Jefferis et al., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

Medium: The term “medium” as used herein means a solution containing nutrients which nourish growing cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. In some embodiments, medium is formulated to a pH and salt concentration optimal for cell survival and proliferation.

Modification: The term “modification” as used herein means an alteration in the physical, chemical, or sequence properties of a protein, polypeptide, antibody, or immunoglobulin. Modifications described herein include amino acid modifications (including amino acid substitutions) and glycoform modifications.

Target Antigen: The term “target antigen” as used herein means the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for the obexelimab is CD19.

Target cell: The term “target cell” as used herein means a cell that expresses a target antigen.

Obexelimab: The term “Obexelimab” as used herein, is an Fc engineered humanized monoclonal antibody (mAb) that binds to the human B-cell restricted surface antigen CD19 and has enhanced Fc binding to Fcγ receptor IIb (FcγRIIb). The molecule is an IgG1 immunoglobulin with a kappa light chain and 2 amino acid substitutions in the constant portion of the heavy chain. Obexelimab is a monoclonal antibody with a projected mass of approximately 147,426 Da based on the amino acid sequence. The heavy and light chains of obexelimab are given by SEQ ID NO: 10, and SEQ ID NO: 9, respectively.

### Anti-CD19 Antibodies and Variants Thereof

Described herein are exemplary anti-CD19 antibodies of the invention, as well as variants thereof. Further, methods described herein can be used for the manufacture of anti-CD19 antibodies as well as variants thereof. Anti-CD19 antibodies (e.g., as described herein) and variants thereof can feature a desirable N-glycan profile or reduced presence of undesirable species.

Exemplary, non-limiting anti-CD19 antibodies, as well as exemplary variants, are described herein.

An exemplary anti-CD19 antibody is obexelimab, which is a monoclonal antibody specific for CD19. Obexelimab exploits the regulation of B-cell receptor (BCR) signaling by FcγRIIb1. Obexelimab binds CD19 of the BCR complex and its Fc is engineered to increase its affinity for the inhibitory FcγRIIb. Since CD19 is associated with the BCR, Obexelimab tethering of CD19 to FcγRIIb on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it does not mediate B cell killing.

In some embodiments, an anti-CD19 antibody (e.g., obexelimab) comprises:
a light chain comprising a variable region having:

a CDR1 comprising
(SEQ ID NO: 2)
RSSKSLQNVNGNTYLY,
a CDR2 comprising
(SEQ ID NO: 3)
RMSNLNS,
and
a CDR3 comprising
(SEQ ID NO: 4)
MQHLEYPIT;

and
a heavy chain comprising a variable region having:

a CDR1 comprising
(SEQ ID NO: 5)
SYVMH,
a CDR2 comprising
(SEQ ID NO: 6)
WIGYINPYNDGTKY,
and
a CDR3 comprising
(SEQ ID NO: 7)
GTYYYGTRVFDY,

wherein the heavy chain comprises amino acid substitutions in the Fc region S267E and L328F.

In some embodiments, comprises amino acid substitutions in the Fc region S267E and L328F as compared to SEQ ID NO: 8:

(SEQ ID NO: 8)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK,

wherein the numbering is according to the EU index, as in Kabat.

In some embodiments, an anti-CD19 antibody (e.g., obexelimab) comprises: a light chain comprising an amino acid sequence of (SEQ ID NO: 9) and heavy chain comprising an amino acid sequence of, (SEQ ID NO: 10), as described in Table 1.

TABLE 1
Sequence of Heavy chain and Light chain
amino acid sequence of an
anti-CD19 antibody obexelimab

| Heavy Chain | Light Chain |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNSG |
| NDGTKYNEKFQGRVTISSDKSISTAYM | VPDRFSGSGSGTEFTLTISSLEPEDFAVYYC |
| ELSSLRSEDTAMYYCARGTYYYGTRVF | MQHLEYPITFGAGTKLEIKRTVAAPSVFIFP |
| DYWGQGTLVTVSSASTKGPSVFPLAPS | PSDEQLKSGTASVVCLLNNFYPREAKVQWKV |
| SKSTSGGTAALGCLVKDYFPEPVTVSW | DNALQSGNSQESVTEQDSKDSTYSLSSTLTL |
| NSGALTSGVHTFPAVLQSSGLYSLSSV | SKADYEKHKVYACEVTHQGLSSPVTKSFNRG |
| VTVPSSSLGTQTYICNVNHKPSNTKVD | EC (SEQ ID NO: 9) |
| KKVEPKSCDKTHTCPPCPAPELLGGPS | |
| VFLFPPKPKDTLMISRTPEVTCVVVDV | |
| EHEDPEVKFNWYVDGVEVHNAKTKPRE | |
| EQYNSTYRVVSVLTVLHQDWLNGKEYK | |
| CKVSNKAFPAPIEKTISKAKGQPREPQ | |
| VYTLPPSREEMTKNQVSLTCLVKGFYP | |
| SDIAVEWESNGQPENNYKTTPPVLDSD | |
| GSFFLYSKLTVDKSRWQQGNVFSCSVM | |
| HEALHNHYTQKSLSLSPGK | |
| (SEQ ID NO: 10) | |

Variable region

| | |
|---|---|
| EVQLVESGGGLVKPGGSLKLSCAASGY | DIVMTQSPATLSLSPGERATLSCRSSKSLQN |
| TFTSYVMHWVRQAPGKGLEWIGYINPY | VNGNTYLYWFQQKPGQSPQLLIYRMSNLNSG |
| NDGTKYNEKFQGRVTISSDKSISTAYM | VPDRFSGSGSGTEFTLTISSLEPEDFAVYYC |
| ELSSLRSEDTAMYYCARGTYYYGTRVF | MQHLEYPITFGAGTKLEIK |
| DYWGQGTLVTVSS | (SEQ ID NO: 11) |
| (SEQ ID NO: 12) | |

In some embodiments, an anti-CD19 antibody (e.g., obexelimab) comprises a light chain variable region comprising SEQ ID NO: 11 and a heavy chain variable region comprising SEQ ID NO: 12.

In some embodiments, an anti-CD19 antibody comprises a heavy chain variable region (VH) and/or a light chain variable region (VL), comprising a CDR1, a CDR2, and a CDR3, each of which differs by no more than 1, 2, 3, 4 or 5 amino acid residues from each of SEQ ID NO: 2, SEQ ID NO: 3, SEQ ID NO: 4, SEQ ID NO: 5, SEQ ID NO: 6 and/or SEQ ID NO: 7.

In some embodiments, an anti-CD19 antibody comprises: a light chain variable region comprising an amino acid sequence that is at least 90%, 91%, 92%, 93%, 94%, 95%, 96%, 97%, 98% or 99% identical to SEQ ID NO: 11. In some embodiments, the variant of obexelimab comprises: a heavy chain variable region comprising an amino acid sequence that is at least 90%, 91%, 92%, 93%, 94%, 95%, 96%, 97%, 98% or 99% identical to SEQ ID NO: 12. In some embodiments, the variant of obexelimab comprises: a light chain variable region comprising an amino acid sequence that is at least 90%, 91%, 92%, 93%, 94%, 95%, 96%, 97%, 98% or 99% identical to SEQ ID NO: 11 and a heavy chain variable region comprising an amino acid sequence that is at least 90%, 91%, 92%, 93%, 94%, 95%, 96%, 97%, 98% or 99% identical to SEQ ID NO: 12, and further comprises amino acid substitutions in the Fc region S267E and L328F as compared to SEQ ID NO: 8, wherein the numbering is according to the EU index.

In some embodiments, an anti-CD19 antibody comprises: a light chain comprising an amino acid sequence 70%, 75%, 80%, 85%, 90%, 95%, 98% or 99% identical to SEQ ID NO: 9. In an embodiment, the variant of obexelimab comprises: a heavy chain comprising an amino acid sequence 70%, 75%, 80%, 85%, 90%, 95%, 98% or 99% identical to SEQ ID NO: 10.

In some embodiments, the variant of obexelimab comprises: a light chain comprising an amino acid sequence that is at least 90%, 91%, 92%, 93%, 94%, 95%, 96%, 97%, 98% or 99% identical to SEQ ID NO: 9 and a heavy chain comprising an amino acid sequence that is at least 90%, 91%, 92%, 93%, 94%, 95%, 96%, 97%, 98% or 99% identical to SEQ ID NO: 10, and the heavy chain of the variant comprises amino acid substitutions in the Fc region S267E and L328F as compared to SEQ ID NO: 8 wherein the numbering is according to the EU index, as in Kabat.

In some embodiments, an anti-CD19 antibody comprises: a light chain comprising an amino acid sequence 70%, 75%, 80%, 85%, 90%, 95%, 98% or 99% identical to SEQ ID NO: 9 and/or amino acid substitutions in the Fc region S267E and L328F as compared to SEQ ID NO: 10, wherein the numbering is according the EU index, as in Kabat.

In some embodiments, an anti-CD19 antibody binds to the same epitope on human CD19, as an antibody comprising a light chain of SEQ ID NO: 9 and a heavy chain of SEQ ID NO: 10. Epitope binding may be determined by a method known in the art.

In some embodiments, an anti-CD19 antibody competes for binding to human CD19, as an antibody comprising a light chain of SEQ ID NO: 9 and a heavy chain of SEQ ID NO: 10, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, monoclonal antibody candidates are screened pairwise for binding to a target protein. Other standard methods involve ELISA-based screens and may require synthesis of sets of overlapping peptides corresponding to the protein of interest.

In some embodiments, an anti-CD19 antibody binds to the extracellular domain of human CD19, wherein the human CD19 comprises an amino acid sequence of SEQ ID NO: 1:

(SEQ ID NO: 1)
MPPPRLLFFLLFLTPMEVRPEEPLVVKVEEGDNAVLQCLKGTSDG
PTQQLTWSRESPLKPFLKLSLGLPGLGIHMRPLAIWLFIFNVSQQ
MGGFYLCQPGPPSEKAWQPGWTVNVEGSGELFRWNVSDLGGLGCG
LKNRSSEGPSSPSGKLMSPKLYVWAKDRPEIWEGEPPCLPPRDSL
NQSLSQDLTMAPGSTLWLSCGVPPDSVSRGPLSWTHVHPKGPKSL
LSLELKDDRPARDMWVMETGLLLPRATAQDAGKYYCHRGNLTMSF
HLEITARPVLWHWLLRTGGWKVSAVTLAYLIFCLCSLVGILHLQR
ALVLRRKRKRMTDPTRRFFKVTPPPGSGPQNQYGNVLSLPTPTSG
LGRAQRWAAGLGGTAPSYGNPSSDVQADGALGSRSPPGVGPEEEE
GEGYEEPDSEEDSEFYENDSNLGQDQLSQDGSGYENPEDEPLGPE
DEDSFSNAESYENEDEELTQPVARTMDFLSPHGSAWDPSREATSL
GSQSYEDMRGILYAAPQLRSIRGQPGPNHEEDADSYENMDNPDGP
DPAWGGGGRMGTWSTR

### N-Glycan Profile

As described herein, exemplary anti-CD19 antibodies (e.g., obexelimab), as well as variants thereof, can feature a desirable N-glycan profile. Different glycosylation patterns of the Fc region of an antibody may impact pharmacokinetic properties of the antibody. In particular, highly sialylated populations of anti-CD19 antibodies as described herein may demonstrate extended serum persistence and thus a more favorable pharmacokinetic profile for therapeutic administration. In contrast, populations of anti-CD19 antibodies possessing N-glycan with high mannose (e.g., mannose-5) may demonstrate accelerated clearance and thus a less favorable pharmacokinetic profile for therapeutic administration. Exemplary N-glycan profiles for anti-CD19 antibodies (e.g., obexelimab) are described herein.

In some embodiments, an anti-CD19 antibody (e.g., obexelimab) is highly sialylated (e.g., comprises an N-glycan profile comprising ≥20% sialylated glycans) and/or has low mannosylation (e.g., comprises an N-glycan profile comprising ≤5% mannose-5 glycans). In embodiments, an anti-CD19 antibody (e.g., obexelimab) is highly sialylated (e.g., comprises an N-glycan profile comprising >20% sialylated glycans). In embodiments, an anti-CD19 antibody (e.g., obexelimab) has low mannosylation (e.g., comprises an N-glycan profile comprising ≤5% mannose-5 glycans). In embodiments, an anti-CD19 antibody (e.g., obexelimab) is highly sialylated (e.g., comprises an N-glycan profile comprising ≥20% sialylated glycans) and has low mannosylation (e.g., comprises an N-glycan profile comprising ≤5% mannose-5 glycans).

In some embodiments, an anti-CD19 antibody (e.g., obexelimab) comprises an N-glycan profile comprising ≥20% sialylated glycans. In some embodiments, an anti-CD19 antibody (e.g., obexelimab) comprises an N-glycan profile comprising ≥21% sialylated glycans. In some embodiments, an anti-CD19 antibody (e.g., obexelimab) comprises an N-glycan profile comprising ≥22% sialylated glycans. In some embodiments, an anti-CD19 antibody comprises an N-glycan profile comprising ≥23% sialylated glycans. In some embodiments, an anti-CD19 antibody (e.g., obexelimab) comprises an N-glycan profile comprising ≥24% sialylated glycans. In some embodiments, an anti-CD19 antibody (e.g., obexelimab) comprises an N-glycan profile comprising ≥25% sialylated glycans. In some embodiments, an anti-CD19 antibody (e.g., obexelimab) comprises an N-glycan profile comprising ≥26% sialylated glycans. In some embodiments, an anti-CD19 antibody (e.g., obexelimab) comprises an N-glycan profile comprising ≥27% sialylated glycans. In some embodiments, an anti-CD19 antibody (e.g., obexelimab) comprises an N-glycan profile comprising ≥28% sialylated glycans. In some embodiments, an anti-CD19 antibody (e.g., obexelimab) comprises an N-glycan profile comprising ≥29% sialylated glycans. In some embodiments, an anti-CD19 antibody (e.g., obexelimab) comprises an N-glycan profile comprising ≥30% sialylated glycans. In some embodiments, an anti-CD19 antibody (e.g., obexelimab) comprises an N-glycan profile comprising ≥31% sialylated glycans. In some embodiments, an anti-CD19 antibody (e.g., obexelimab) comprises an N-glycan profile comprising ≥32% sialylated glycans.

In some embodiments, an anti-CD19 antibody (e.g., obexelimab) comprises an N-glycan profile comprising ≤5% mannose-5 glycans. In some embodiments, an anti-CD19 antibody (e.g., obexelimab) comprises an N-glycan profile comprising ≤4% mannose-5 glycans. In some embodiments, an anti-CD19 antibody (e.g., obexelimab) comprises an N-glycan profile comprising ≤3% mannose-5 glycans. In some embodiments, an anti-CD19 antibody (e.g., obexelimab) comprises an N-glycan profile comprising ≤2% mannose-5 glycans. In some embodiments, an anti-CD19 antibody (e.g., obexelimab) comprises an N-glycan profile comprising ≤1% mannose-5 glycans. In some embodiments, an anti-CD19 antibody (e.g., obexelimab) comprises an N-glycan profile comprising ≤0.9% mannose-5 glycans. In some embodiments, an anti-CD19 antibody (e.g., obexelimab) comprises an N-glycan profile comprising ≤0.8% mannose-5 glycans. In some embodiments, an anti-CD19 antibody (e.g., obexelimab) comprises an N-glycan profile comprising ≤0.7% mannose-5 glycans. In some embodiments, an anti-CD19 antibody (e.g., obexelimab) comprises an N-glycan profile comprising ≤0.6% mannose-5 glycans. In some embodiments, an anti-CD19 antibody (e.g., obexelimab) comprises an N-glycan profile comprising ≤0.5% mannose-5 glycans. In some embodiments, an anti-CD19 antibody (e.g., obexelimab) comprises an N-glycan profile comprising ≤0.4% mannose-5 glycans. In some embodiments, an anti-CD19 antibody (e.g., obexelimab) comprises an N-glycan profile comprising ≤0.3% mannose-5 glycans. In some embodiments, an anti-CD19 antibody (e.g., obexelimab) comprises an N-glycan profile comprising ≤0.2% mannose-5 glycans.

In some embodiments, an anti-CD19 antibody (e.g., obexelimab) comprises an N-glycan profile comprising ≥20% sialylated glycans and ≤5% mannose-5 glycans. In some embodiments, an anti-CD19 antibody (e.g., obexelimab) comprises an N-glycan profile comprising ≥25% sialylated glycans and ≤1% mannose-5 glycans. In some embodiments, an anti-CD19 antibody (e.g., obexelimab) comprises an N-glycan profile comprising ≥30% sialylated glycans and ≤0.5% mannose-5 glycans. In some embodiments, an anti-CD19 antibody (e.g., obexelimab) comprises an N-glycan profile comprising ≥32% sialylated glycans and ≤0.2% mannose-5 glycans.

In some embodiments, an anti-CD19 antibody (e.g., obexelimab) comprises an N-glycan profile comprising 20% to 35% sialyated glycans. In some embodiments, an anti-CD19 antibody (e.g., obexelimab) comprises an N-glycan profile comprising 25% to 35% sialyated glycans. In some embodiments, an anti-CD19 antibody (e.g., obexelimab) comprises an N-glycan profile comprising 30% to 35% sialyated glycans.

In some embodiments, an anti-CD19 antibody (e.g., obexelimab) comprises an N-glycan profile comprising 0.2% to 5% mannose-5 glycans. In some embodiments, an anti-CD19 antibody (e.g., obexelimab) comprises an N-glycan profile comprising 0.2% to 1% mannose-5 glycans. In some embodiments, an anti-CD19 antibody (e.g., obexelimab) comprises an N-glycan profile comprising 0.2% to 0.5% mannose-5 glycans.

In some embodiments, an anti-CD19 antibody (e.g., obexelimab) comprises an N-glycan profile comprising 20% to 35% GOF glycans. In some embodiments, an anti-CD19 antibody (e.g., obexelimab) comprises an N-glycan profile comprising 20% to 25% G1Fa glycans. In some embodiments, an anti-CD19 antibody (e.g., obexelimab) comprises an N-glycan profile comprising 20% to 25% G1Fa glycans. In some embodiments, an anti-CD19 antibody (e.g., obexelimab) comprises an N-glycan profile comprising 5% to 7% G1Fb glycans. In some embodiments, an anti-CD19 antibody (e.g., obexelimab) comprises an N-glycan profile comprising 13% to 22% G2F/GIFS1a glycans. In some embodiments, an anti-CD19 antibody (e.g., obexelimab) comprises an N-glycan profile comprising 1% to 2% GIFS1b glycans. In some embodiments, an anti-CD19 antibody (e.g., obexelimab) comprises an N-glycan profile comprising 0.1% to 1% GIFS1+GN glycans. In some embodiments, an anti-CD19 antibody (e.g., obexelimab) comprises an N-glycan profile comprising 5% to 13% G2FS1 glycans. In some embodiments, an anti-CD19 antibody (e.g., obexelimab) comprises an N-glycan profile comprising 5% to 12% G2FS2 glycans.

In some embodiments, an anti-CD19 antibody (e.g., obexelimab) comprises an N-glycan profile as depicted in Table 4.

### Variants

Methods described herein can also be suitable for the preparation of variants of anti-CD19 antibodies, including the exemplary variants of obexelimab as described herein.

In some embodiments, a variant of obexelimab is an immunoglobulin specific for CD19 that comprises: a light chain comprising a variable region having a CDR1 comprising SEQ ID NO: 2, a CDR2 comprising SEQ ID NO: 3, and a CDR3 comprising SEQ ID NO: 4; and a heavy chain comprising a variable region having a CDR1 comprising SEQ ID NO: 5, a CDR2 comprising SEQ ID NO: 6, and a CDR3 comprising SEQ ID NO: 7, wherein the heavy chain comprises amino acid substitutions in the Fc region S267E and L328F as compared to SEQ ID NO: 8, wherein the numbering is according to the EU index, as in Kabat.

### Purity-Low- and High-Molecular Weight Structural Species

Antibody production can result in the formation of structural species, such as high molecular weight (HMW) species and low molecular weight (LMW) species. HMW species may comprise aggregated forms of the antibody, while LMW species may comprise truncated fragments such as heavy-heavy-light chain variants. The presence of these heterogenous structural species can impact the immunogenicity or pharmacokinetic properties of a therapeutic, and thus it is desirable to minimize their relative abundance.

In some embodiments, an anti-CD19 antibody described herein is substantially free (e.g., ≤20, 15, 10, or 5%) of high molecular weight species and/or substantially free (e.g., ≤20, 15, 10, or 5%) of low molecular weight species. In embodiments, an anti-CD19 antibody described herein is substantially free (e.g., ≤20, 15, 10, or 5%) of high molecular weight species. In embodiments, an anti-CD19 antibody described herein is substantially free (e.g., ≤20, 15, 10, or 5%) of low molecular weight species. In embodiments, an anti-CD19 antibody described herein is substantially free (e.g., ≤20, 15, 10, or 5%) of high molecular weight species and substantially free (e.g., ≤20, 15, 10, or 5%) of low molecular weight species.

The amount of high molecular weight and low molecular weight species can be determined according to any suitable method in the art.

In some embodiments, the presence of low molecular weight species is measured by SE-UPLC. In some embodiments, ≤5% of an anti-CD19 antibody (e.g., obexelimab) is present as low molecular weight species. In some embodiments, ≤4% of an anti-CD19 antibody (e.g., obexelimab) is present as low molecular weight species. In some embodiments, ≤3% of an anti-CD19 antibody (e.g., obexelimab) is present as low molecular weight species. In some embodiments, ≤2% of an anti-CD19 antibody (e.g., obexelimab) is present as low molecular weight species. In some embodiments, ≤1.8% of an anti-CD19 antibody (e.g., obexelimab) is present as low molecular weight species. In some embodiments, ≤1.6% of an anti-CD19 antibody (e.g., obexelimab) is present as low molecular weight species. In some embodiments, ≤1.4% of an anti-CD19 antibody (e.g., obexelimab) is present as low molecular weight species. In some embodiments, ≤1.2% of an anti-CD19 antibody (e.g., obexelimab) is present as low molecular weight species. In some embodiments, ≤1% of an anti-CD19 antibody (e.g., obexelimab) is present as low molecular weight species. In some embodiments, ≤0.9% of an anti-CD19 antibody (e.g., obexelimab) is present as low molecular weight species. In some embodiments, ≤0.8% of an anti-CD19 antibody (e.g., obexelimab) is present as low molecular weight species. In some embodiments, ≤0.7% of an anti-CD19 antibody (e.g., obexelimab) is present as low molecular weight species. In some embodiments, ≤0.6% an the anti-CD19 antibody (e.g., obexelimab) is present as low molecular weight species. In some embodiments, ≤0.5% of an anti-CD19 antibody (e.g., obexelimab) is present as low molecular weight species. In some embodiments, ≤0.4% of an anti-CD19 antibody (e.g., obexelimab) is present as low molecular weight species. In some embodiments, ≤0.3% of an anti-CD19 antibody (e.g., obexelimab) is present as low molecular weight species.

In some embodiments, the presence of high molecular weight species is measured by SE-UPLC. In some embodiments, ≤5% of an anti-CD19 antibody (e.g., obexelimab) is present as high molecular weight species. In some embodiments, ≤4% of an anti-CD19 antibody (e.g., obexelimab) is present as high molecular weight species. In some embodiments, ≤3% of an anti-CD19 antibody (e.g., obexelimab) is present as high molecular weight species. In some embodiments, ≤2% of an anti-CD19 antibody (e.g., obexelimab) is present as high molecular weight species. In some embodiments, ≤1.8% of an anti-CD19 antibody (e.g., obexelimab) is present as high molecular weight species In some embodiments, ≤1.6% of an anti-CD19 antibody (e.g., obexelimab) is present as high molecular weight species. In some embodiments, ≤1.4% of an anti-CD19 antibody (e.g., obexelimab) is present as high molecular weight species. In some embodiments, ≤1.2% of an anti-CD19 antibody (e.g., obexelimab) is present as high molecular weight species. In some embodiments, ≤1% of an anti-CD19 antibody (e.g., obexelimab) is present as high molecular weight species. In some embodiments, ≤0.9% of an anti-CD19 antibody (e.g., obexelimab) is present as high molecular weight species. In some embodiments, ≤0.8% of an anti-CD19 antibody (e.g., obexelimab) is present as high molecular weight species. In some embodiments, ≤0.7% of an anti-CD19 antibody (e.g., obexelimab) is present as high molecular weight species. In some embodiments, ≤0.6% of an anti-CD19 antibody (e.g., obexelimab) is present as high molecular weight species. In some embodiments, ≤0.5% of an anti-CD19 antibody (e.g., obexelimab) is present as high molecular weight species.

### Fc Receptor Binding Properties

Anti-CD19 antibodies (e.g., obexelimab) disclosed herein can comprise an Fc variant that has enhanced Fc binding to the inhibitory Fcγ receptor IIb (FcγRIIb). FcγRIIb, the only FcR on B cells, serves as an antibody-sensing down-regulator of humoral immunity that is naturally engaged by immune complexes. When sufficient antibody is raised against a given antigen, specific immune complexes form and co-engage FcγRIIb and the B cell receptor (BCR) with high avidity, selectively suppressing only B cells recognizing cognate antigen. In addition, FcγRIIb regulates the activity of other B cell stimulators including interleukin (IL)-4, LPS, and BAFF that amplify BCR-driven proliferation and differentiation. By simultaneously binding CD19 and FcγRIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIIb. In other embodiments, immunoglobulins disclosed herein provide enhanced affinity for FcγRIIb, yet reduced affinity for one or more activating FcγRs, including for example FcγRI, FcγRIIa, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIIb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from about two to fifty amino acid modifications, e.g., from about two to ten amino acid modifications, from about two to about five amino acid modifications, etc. compared to the parent. Thus, the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5th Ed., United States Public Health Service, National Institutes of Health, Bethesda, hereby entirely incorporated by reference). In brief, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. Immunoglobulins may also comprise sequences from other immunoglobulin classes such as IgA, IgE, IgGD, IgGM, and the like. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. To establish homology, the amino acid sequence of a first IgG outlined herein can be directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, Con C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human lgG1, by using the methods described above or other methods for determining equivalent residues, the variant antibody may be engineered in another lgG1 parent antibody that binds a different antigen, a human lgG2 parent antibody, a human IgA parent antibody, a mouse lgG2a or lgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein, is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) lgG1 binds FcγRIIb with an affinity of about 1.5 mM, or about 1500 nM. Furthermore, some Fc variants described herein bind FcγRIIb with an affinity about 10-fold greater to WT lgG1. As disclosed herein, greater or enhanced affinity means having a KD lower than about 100 nM, for example between about 10 nM-about 100 nM, between about 1-about 100 nM, or less than about 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136).

An important parameter that determines the most beneficial selectivity of a given Fc variant to treat a given disease is the context of the Fc variant. Thus, the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Compositions of the Anti-CD19 Antibody

The present invention provides compositions of anti-CD19 antibodies (e.g., obexelimab). In some embodiments, the composition is a liquid composition.

In some embodiments, the liquid composition comprises an anti-CD19 antibody (e.g., obexelimab) at various concentrations. In some embodiments, the liquid composition may contain an anti-CD19 antibody at a concentration ranging between about 10-300 mg/ml (e.g., about 10-250 mg/ml, about 10-200 mg/ml, about 10-180 mg/ml, about 10-160 mg/ml, about 10-150 mg/ml, about 10-140 mg/ml, about 10-130 mg/ml, about 10-125 mg/ml, about 100-125 mg/ml, about 100-180 mg/ml, about 100-150 mg/ml, about 100-130 mg/ml, about 100-125 mg/ml, about 100-170 mg/ml, about 100-160 mg/ml, about 100-150 mg/ml, about 100-200 mg/ml, about 120-130 mg/ml). In some embodiments, an anti-CD19 antibody (e.g., obexelimab) is present at a concentration ranging from 100-150 mg/ml. In some embodiments, an anti-CD19 antibody (e.g., obexelimab) is present at a concentration ranging from 113-138 mg/ml.

Many therapeutic agents, and in particular the antibodies of the present invention, require controlled pH and specific excipients to maintain their solubility and stability in the compositions of the present invention.

The pH of the liquid composition is an additional factor which is capable of altering the solubility of an anti-CD19 antibody (e.g., obexelimab) in an aqueous composition. In some embodiments, compositions of the present invention contain one or more buffers. In some embodiments, compositions according to the invention contain an amount of buffer sufficient to maintain the optimal pH of said composition between about 4.0-8.0, between about 5.0-7.5, between about 5.5-7.0, between about 6.0-7.0 and between about 6.0-7.5. In some embodiments the liquid composition has a pH ranging from 5 to 6.

In some embodiments, compositions as described herein are in a liquid or aqueous form. In some embodiments, a composition is lyophilized. Such lyophilized compositions may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

Compositions described herein may be characterized by their tolerability. As used herein, the terms “tolerable” and “tolerability” refer to the ability of the compositions of the present invention to not elicit an adverse reaction in the patient to whom such composition is administered, or alternatively not to elicit a serious adverse reaction in the patient to whom such composition is administered. In some embodiments, a composition described herein is well tolerated by the patient to whom such composition is administered.

The amounts of anti-CD19 antibody (e.g., obexelimab), buffer, tonicity modifier, solvent, and surfactants may vary. In some embodiments, a liquid composition comprises obexelimab, a buffer, and a tonicity modifier. In some embodiments, a liquid composition comprises 100 mg/mL to 250 mg/mL obexelimab, an acetate buffer, and proline. In some embodiments, a liquid composition comprises 125 mg/mL obexelimab, an acetate buffer, and proline. In some embodiments, a liquid composition comprises 100 mg/mL to 250 mg/mL obexelimab, 5 to 40 mM acetate buffer, and 1% to 5% (w/v) proline. In some embodiments, a liquid composition comprises 125 mg/mL obexelimab, 20 mM acetate buffer, and 3% (w/v) proline, at pH 5 to 6. In some embodiments, a liquid composition comprises 125 mg/mL obexelimab, 20 mM acetate buffer, and 3% (w/v) proline at pH 5.5. In some embodiments, a liquid composition comprises 125 mg/mL obexelimab, 2.35 mg/mL sodium acetate trihydrate, 0.17 mg/mL acetic acid (at density 1.053 g/mL), 30 mg/mL L-proline, 0.1 mg/mL polysorbate 80, pH 5.5. In some embodiments, a liquid composition has an osmolality of 265 to 441 mOsm/kg. In some embodiments, a liquid composition has a viscosity less than 10 cP at 25° C.

### Manufacturing Processes & Conditions

Described herein are methods of manufacturing anti-CD19 antibodies or variants thereof, including as described herein. For example, methods described herein can be used to manufacture any anti-CD19 antibody described herein, or a variant thereof.

In one aspect, the invention features a method of manufacturing an anti-CD19 antibody (e.g., obexelimab), where the method comprises culturing mammalian cells engineered to express the anti-CD19 antibody. In embodiments, the culturing comprises a fed-batch production process. In other embodiments, the culturing is under conditions such that cell viability ≥95% is maintained (e.g., during a fed-batch production process).

One skilled in the art will appreciate that a variety of methods known in the art may be employed to expand the cells or population of cells (e.g., mammalian cells) engineered to express an anti-CD19 antibody (e.g., obexelimab) to a sufficient volume to practice a method described herein. For example, following engineering of the cells to express an anti-CD19 antibody (e.g., obexelimab), the cells may be then subject to single cell cloning and expanded via serial passage. Aliquots of the expanded engineered cells may then be cryopreserved in vials for subsequent use in a production process. These cryopreserved vials may then be expanded via serial passage under conditions sufficient to maintain cell growth to achieve the necessary number of cells to enter a production stage, which may also be referred to as a production process. One skilled in the art will appreciate that a production process may be seeded at a range of densities to practice a method described herein. In some embodiments, a production process is seeded at a target viable cell density of 0.3×10⁶ cells/mL to 0.5×10⁶ cells/mL. In some embodiments, a production process is seeded at a target viable cell density of 0.4×10⁶ cells/mL.

### Engineered Mammalian Cells

In some embodiments, methods of manufacturing anti-CD19 antibodies, or variants thereof, comprise culturing mammalian cells engineered to express the anti-CD19 antibody.

Mammalian cells or cell types suitable to cell culture, and to expression of polypeptides, may be utilized in accordance with the present invention as a host cell. Non-limiting examples of mammalian cells that may be used in accordance with the present invention include human embryonic kidney 293 cells (HEK293), HeLa cells; BALB/c mouse myeloma line (NSO/1, ECACC No: 851 10503); human retinoblasts (PER.C6 (CruCell, Leiden, The Netherlands); monkey kidney CV1 line transformed by SV40 (COS-7, ATCC CRL 1651); human embryonic kidney line (293 or 293 cells sub cloned for growth in Suspension culture, Graham et al., J. Gen Virol. 36:59 (1977)); baby hamster kidney cells (BHK, ATCC CCL 10); Chinese hamster ovary cells+/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCCCCL70); African green monkey kidney cells (VERO 76, ATCC CRL-1 587); human cervical carcinoma cells (HeLa, ATCC CCL 2); canine kidney cells (MDCK, ATCC CCL34); buffalo rat liver cells (BRL3A, ATCCCRL1442): human lung cells (W138, ATCC CCL 75); human liver cells (Hep G2, HB 8065); mouse mammary tumor (MMT 060562, ATCCCCL51); TR1 cells (Mather et al., Annals N.Y. Acad. Sci., 383:44-68 (1982)); MRC5 cells; FS4 cells; and a human hepatoma line (Hep G2). In embodiments, engineered mammalian cells are Chinese hamster ovary (CHO) cells.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

Briefly, host cells of the present invention are engineered to stably express an anti-CD19 antibody (e.g., obexelimab). One skilled in the art will appreciate that a variety of methods may be used to engineer the host cells of the present invention, including but not limited to transfection, transduction, or transformation of the host cell with one or more vectors expressing the heavy chain and light chain of the anti-CD19 antibody (e.g., obexelimab). Examples of transformation, transfection, and transduction methods, which are well known in the art, include liposome delivery, i.e., Lipofectamine™ (Gibco BRL) Method of Hawley-Nelson, Focus 15:73 (1193), electroporation, CaPO delivery method of Graham and van der Erb, Virology, 52:456-457 (1978), DEAE-Dextran medicated delivery, microinjection, biolistic particle delivery, polybrene mediated delivery, cationic mediated lipid delivery, transduction, and viral infection, such as, e.g., retrovirus, lentivirus, adenovirus, adeno-associated virus and Baculovirus (Insect cells). Once introduced inside cells, expression vectors may be integrated stably in the genome or exist as extra-chromosomal constructs. Vectors may also be amplified and multiple copies may exist or be integrated in the genome. One skilled in the art will further appreciate that a variety of methods known in the art may be employed to screen and select an engineered clonal cell line or a population of cells suitable for use in a method described herein.

In some embodiments, the host cells comprise mammalian cells engineered to express an anti-CD19 antibody (e.g., obexelimab). In some embodiments, engineered mammalian cells comprise suspension adapted mammalian cells (e.g., any mammalian cell as described herein). In some embodiments, engineered mammalian cells comprise CHO cells. In some embodiments, CHO cells engineered to express an anti-CD19 antibody (e.g., obexelimab) express the anti-CD19 antibody at more than one integration site.

Various cell culture medium and conditions may be used to produce an anti-CD19 antibody (e.g., obexelimab) according to a method described herein. For example, an anti-CD19 antibody (e.g., obexelimab) may be produced in serum-containing or serum-free medium. In some embodiments, an anti-CD19 antibody (e.g., obexelimab) is produced in serum-free medium. In some embodiments, an anti-CD19 antibody (e.g., obexelimab) is produced in an animal free medium, i.e., a medium that lacks animal-derived components. In some embodiments, an anti-CD19 antibody (e.g., obexelimab) is produced in a chemically defined medium. As used herein, the term “chemically-defined nutrient medium” refers to a medium of which substantially all of the chemical components are known. In some embodiments, a chemically defined nutrient medium is free of animal-derived components such as serum, serum derived proteins (e.g., albumin or fetuin), and other components. In some cases, a chemically defined medium comprises one or more proteins (e.g., protein growth factors or cytokines) In some cases, a chemically defined nutrient medium comprises one or more protein hydrolysates. In other cases, a chemically-defined nutrient medium is a protein-free media, i.e., a serum-free media that contains no proteins, hydrolysates or components of unknown composition. In some embodiments, a chemically defined medium may be supplemented by one or more animal derived components. Such animal derived components include, but are not limited to, fetal calf serum, horse serum, goat serum, donkey serum, human serum, and serum derived proteins such as albumins (e.g., bovine serum albumin or human serum albumin). In some embodiments, dissolved oxygen (DO) is controlled to approximately 40% during the production process. In some embodiments, pH is controlled to approximately 7.0 during the production process.

Various cell culture mediums and processes may be used to produce an anti-CD19 antibody (e.g., obexelimab) as described herein. Such processes include, but are not limited to, roller bottle processes, bioreactor batch processes, bioreactor fed-batch processes, and bioreactor perfusion processes. In some embodiments, a production process is performed at a scale of at least 10 L, at least 100 L, at least 200 L, at least 500 L, at least 1000 L, at least 1500 L, or at least 2000 L.

In some embodiments, a production process as described herein comprises a perfusion process. A perfusion process refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified. Typically, a cell culture process involving a perfusion process is referred to as “perfusion culture.” Typically, nutritional Supplements are provided in a fresh medium during a perfusion process. In some embodiments, a fresh medium may be identical or similar to the base medium used in the fed-batch process.

In some embodiments, a production process as described herein comprises a fed-batch process: The term fed-batch process as used herein refers to a method of culturing cells in which additional components are provided to the culture at some time subsequent to the beginning of the culture process. The provided components typically comprise nutritional supplements for the cells which have been depleted during the culturing process. A fed-batch culture is typically stopped at some point and the cells and/or components in the medium are harvested and optionally purified.

In some embodiments, methods of manufacturing an anti-CD19 antibody maintain high cell viability during the production process. In embodiments, high cell viability is maintained during the production process until the harvest of the cell culture. One skilled in the art will appreciate that a variety of methods may be used to measure cell viability including, for example, trypan blue dye exclusion or luminogenic ATP assays.

In some embodiments, a method of manufacturing an anti-CD19 antibody (e.g., obexelimab) comprises culturing engineered mammalian cells engineered to express the anti-CD19 antibody in a production process under conditions such that cell viability ≥80% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥85% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥90% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥95% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥96% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥97% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥98% is maintained during the production process. In some embodiments, a method of manufacturing an anti-CD19 antibody (e.g., obexelimab) comprises culturing engineered mammalian cells engineered to express the anti-CD19 antibody in a production process under conditions such that cell viability ≥99% is maintained during the production process.

In some embodiments, a method of manufacturing an anti-CD19 antibody (e.g., obexelimab) comprises culturing engineered mammalian cells engineered to express the anti-CD19 antibody in a fed-batch production process under conditions such that cell viability ≥80% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥85% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥90% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability 95% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥96% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥97% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥98% is maintained during the production process. In some embodiments, the method of manufacturing an anti-CD19 antibody (e.g., obexelimab) comprises culturing engineered mammalian cells engineered to express the anti-CD19 antibody in a fed-batch production process under conditions such that cell viability ≥99% is maintained during the production process.

In some embodiments, a production process is performed at a scale of at least 10 L, at least 100 L, at least 200 L, at least 500 L, at least 1000 L, at least 1500 L, or at least 2000 L. In embodiments, a production process is a fed-batch production process. In some embodiments, a fed-batch production process is performed at a scale of at least 10 L, at least 100 L, at least 200 L, at least 500 L, at least 1000 L, at least 1500 L, or at least 2000 L. In some embodiments, a fed-batch production process is performed at a scale of at least 10 L. In some embodiments, a fed-batch production process is performed at a scale of at least 100 L. In some embodiments, a fed-batch production process is performed at a scale of at least 200 L. In some embodiments, a fed-batch production process is performed at a scale of at least 500 L. In some embodiments, a fed-batch production process is performed at a scale of at least 1000 L. In some embodiments, a fed-batch production process is performed at a scale of at least 1500 L. In some embodiments, a fed-batch production process is performed at a scale of at least 2000 L.

In some embodiments, a method described herein comprises controlling the amount of lactate dehydrogenase (LDH) during the production process. LDH is released into cell culture at increasing concentrations as the plasma membrane of cells becomes damaged, and thus LDH may be used to assess the amount of damaged or dead cells in a cell culture. In some embodiments, a method comprises controlling lactate dehydrogenase (LDH)<1500 U/L during the fed-batch process. In some embodiments, a method comprises controlling lactate dehydrogenase (LDH)<1000 U/L during the fed-batch process. In some embodiments, a method comprises controlling lactate dehydrogenase (LDH)<500 U/L during the fed-batch process.

In some embodiments, a method described herein comprises one or more steps of supplementing nutrients depleted during the culturing process. In some embodiments, a method comprises a step of feeding cells with one or more of glutamine, galactose, asparagine, serine, tyrosine, and/or cystine.

In some embodiments, a method comprises a step of feeding cells with glutamine (e.g., L-glutamine).

In some embodiments, a method comprises a step of feeding cells with galactose (e.g., L-galactose).

In some embodiments, a method comprises a step of feeding cells with asparagine (e.g., L-asparagine).

In some embodiments, a method comprises a step of feeding cells with serine (e.g., L-serine).

In some embodiments, a method comprises a step of feeding cells with tyrosine (e.g., L-tyrosine).

In some embodiments, a method comprises a step of feeding cells with cystine (e.g., L-cystine).

In some embodiments, a method comprises a step of providing cells with glucose (e.g., D-glucose).

In some embodiments, a method comprises a step of feeding cells during a fed-batch production process.

In some embodiments, a method comprises a step of feeding cells with glutamine at day 2 of the fed-batch process.

In some embodiments, a method comprises a step of feeding cells with galactose at day 2 of the fed-batch process.

In some embodiments, a method comprises a step of feeding cells with asparagine at day 2 of the fed-batch process.

In some embodiments, a method comprises a step of feeding cells with serine at day 2 of the fed-batch process.

In some embodiments, a method comprises a step of feeding cells with tyrosine at day 2 of the fed-batch process.

In some embodiments, a method comprises a step of feeding cells with cystine at day 2 of the fed-batch process.

In some embodiments, a method comprises a step of feeding cells with galactose between day 6 and day 11 of the fed-batch process.

In some embodiments, a method comprises a step of feeding cells with galactose when glucose≤5.5 g/L.

In some embodiments, a method comprises a step of providing glucose when glucose≤4.5 g/L.

In some embodiments, a method comprises an exemplary nutrient feeding schedule comprising one or more steps of those described in Table 3 herein. In embodiments, an exemplary nutrient feeding schedule comprises each step described in Table 3 herein.

In some embodiments, methods described herein comprise controlling the partial pressure of CO₂ (pCO2) during the production process (e.g., a fed batch process). In such methods, controlling pCO2 can influence the rate of cell growth of mammalian cell culture and influence product quality attributes such as glycosylation patterns in antibody producing mammalian cells. In some embodiments, a method comprises a step of controlling pCO2 to ≥80 mmHg during a fed-batch production process. In some embodiments, a method comprises a step of controlling pCO2 to ≤140 mmHg during a fed-batch production process. In some embodiments, a method comprises a step of controlling pCO2 between 70 to 140 mmHg during a fed-batch production process. In some embodiments, a method comprises a step of controlling pCO2 between 80 to 130 mmHg during a fed-batch production process. In some embodiments, a method comprises a step of controlling pCO2 between 80 to 130 mmHg between day 4 to day 10 of the fed-batch process.

In some embodiments, a method comprises a step of controlling pCO2 to ≥80 mmHg between day 4 and day 10 of a production process (e.g., a fed-batch production process). In some embodiments, a method comprises a step of controlling pCO2 to ≤140 mmHg between day 4 and day 10 of a fed-batch production process. In some embodiments, a method comprises a step of controlling pCO2 between 70 to 140 mmHg between day 4 and day 10 of a fed-batch production process. In some embodiments, a method comprises a step of controlling pCO2 between 80 to 130 mmHg between day 4 and day 10 of a fed-batch production process.

In some embodiments, methods described herein comprise controlling the temperature of a production process (e.g., a fed-batch production process). In some embodiments, a method comprises culturing the mammalian cells at a target temperature of 37° C. during a fed-batch production process. In some embodiments, a method comprises shifting the mammalian cells from a target temperature of 37° C. to a target temperature of 34° C. during a fed-batch production process. In some embodiments, a method comprises shifting the mammalian cells from a target temperature of 37° C. to a target temperature of 34° C. when a fed-batch production process exceeds a cell density of 5.0×10⁶ cells/mL. In some embodiments, a method comprises a step of shifting the mammalian cells from a target temperature of 37° C. to a target temperature of 34° C. when a fed-batch production process exceeds a cell density of 5.0×10⁶ cells/mL target temperature between day 3 and day 5 of the fed-batch production process.

### Purification of Anti-CD19 Antibodies

Various methods may be used to purify or isolate an anti-CD19 antibody (e.g., obexelimab) produced according to methods as described herein. In some embodiments, host cells expressing an anti-CD19 antibody (e.g., obexelimab) are lysed for purification. Lysis of mammalian host cells can be achieved by any number of means well known to those of ordinary skill in the art, including physical disruption by glass beads and exposure to high pH conditions. An anti-CD19 antibody (e.g., obexelimab) may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987: Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methylsulfonyl fluoride (PMSF), leupeptin, pepstatin, or aprotinin may be added at any or all stages in order to reduce or eliminate degradation of the polypeptide or protein during the purification process. Protease inhibitors are particularly desired when cells must be lysed in order to isolate and purify the expressed polypeptide or protein.

In an exemplary method of purifying an anti-CD19 antibody (e.g., obexelimab), the production process is harvested when cell viability is ≥97%. The bulk harvest is then clarified via depth filtration, with the clarified bulk harvest then loaded onto an equilibrated Protein A Affinity column before proceeding to low pH viral inactivation and intermediate depth filtration. The resulting intermediate is then further purified through ion exchange chromatography prior to viral filtration and ultrafiltration/diafiltration.

### Characterization of the Anti-CD19 Antibody Composition

An anti-CD19 antibody (e.g., obexelimab) as described herein may be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion high-performance liquid chromatography (SE-HPLC), size exclusion ultra-performance liquid chromatography (SE-UPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, capillary isoelectric focusing (cIEF), CIEF-desialylation, and combinations thereof. In some embodiments, evaluation of an anti-CD19 antibody, or a composition thereof, may include a step of evaluating appearance (either liquid or cake appearance).

In some embodiments, an anti-CD19 antibody of the present invention comprises a specific N-glycan profile. One skilled in the art will appreciate that the N-gylcan profile of an antibody may be characterized through a variety of methods. One exemplary method of characterizing the N-glycan profile of an antibody is through Ultra High Performance Liquid Chromatography Fluorescent Light Detection (UPLC-FLD). UPLC-FLD may be performed through glycan release, purification, and labeling followed by detection analysis.

In some embodiments, a composition of the present invention comprises a limited residual amount of host cell protein (HCP). One skilled in the art will appreciate that a variety of methods may be used to assess the amount of HCP in a composition, such as through ELISA. In some embodiments, a composition comprises ≤100 ng/mg host cell protein (HCP). In some embodiments, a composition comprises ≤75 ng/mg host cell protein (HCP). In some embodiments, a composition comprises ≤50 ng/mg host cell protein (HCP). In some embodiments, a composition comprises ≤30 ng/mg host cell protein (HCP). In some embodiments, a composition comprises ≤25 ng/mg host cell protein (HCP). In some embodiments, a composition comprises ≤20 ng/mg host cell protein (HCP). In some embodiments, a composition comprises ≤15 ng/mg host cell protein (HCP). In some embodiments, a composition comprises ≤10 ng/mg host cell protein (HCP).

In some embodiments, a composition as described herein comprises a particular charge profile. Various methods may be used to determine the charge profile of an antibody composition, such as Imaged Capillary Isoelectric Focusing (icIEF). The presence of a high level of sialylated glycans may make it difficult to reveal the charge variants derived from the side chains of amino acid residues, and thus the sialic acid of the composition may be removed prior to testing, and thus a modified Imaged Capillary Isoelectric Focusing with Desialylation Treatment (icIEF-Desialylation) may be used. One skilled in the art will further appreciate that a variety of methods may be used to remove sialic acid prior to testing, for example, through the use of Sialidase A. In some embodiments, a composition comprises a main charge variant that constitutes ≥28% of charge variants by icIEF-Desialylation. In some embodiments, a composition comprises a main charge variant that constitutes ≥30% of charge variants by icIEF-Desialylation. In some embodiments, a composition comprises a main charge variant that constitutes ≥40% of charge variants by icIEF-Desialylation. In some embodiments, a composition comprises a main charge variant that constitutes ≥50% of charge variants by icIEF-Desialylation. In some embodiments, a composition comprises an acidic charge variant that constitutes ≤30% of charge variants by icIEF-Desialylation. In some embodiments, a composition comprises an acidic charge variant that constitutes ≤25% of charge variants by icIEF-Desialylation. In some embodiments, a composition comprises an acidic charge variant that constitutes ≤20% of charge variants by icIEF-Desialylation. In some embodiments, a composition comprises a basic charge variant that constitutes ≤30% of charge variants by icIEF-Desialylation. In some embodiments, a composition comprises a basic charge variant that constitutes ≤25% of charge variants by icIEF-Desialylation. In some embodiments, a composition comprises a basic variant that constitutes ≤20% of charge variants by icIEF-Desialylation.

In some embodiments, a composition as described herein comprises an anti-CD19 antibody (e.g., obexelimab) in a predominately monomer form (e.g., substantially free of HMW and/or LMW species). One skilled in the art will appreciate that a variety of methods may be used to measure the monomer, HMW species and LMW species, for example, through Size exclusion Ultra Performance Liquid Chromatography (SE-UPLC). In some embodiments, a composition comprises a main monomer peak that constitutes ≥95% of the total antibody (e.g., as measured by SE-UPLC Purity). In some embodiments, a composition comprises a main monomer peak that constitutes ≥96% of the total antibody (e.g., as measured by SE-UPLC Purity). In some embodiments, a composition comprises a main monomer peak that constitutes ≥97% of the total antibody (e.g., as measured by SE-UPLC Purity). In some embodiments, a composition comprises a main monomer peak that constitutes ≥98% of the total antibody (e.g., as measured by SE-UPLC Purity). In some embodiments, a composition comprises high molecular weight species that constitute ≤5.0% of the total antibody (e.g., as measured by SE-UPLC Purity). In some embodiments, a composition comprises high molecular weight species that constitute ≤2.0% of the total antibody (e.g., as measured by SE-UPLC Purity). In some embodiments, a composition comprises high molecular weight species that constitute ≤1.0% of the total antibody (e.g., as measured by SE-UPLC Purity). In some embodiments, a composition comprises low molecular weight species that constitute ≤5.0% of the total antibody (e.g., as measured by SE-UPLC Purity). In some embodiments, a composition comprises low molecular weight species that constitute ≤2.0% of the total antibody (e.g., as measured by SE-UPLC Purity). In some embodiments, a composition comprises low molecular weight species that constitute ≤1.0% of the total antibody (e.g., as measured by SE-UPLC Purity).

In some embodiments, a composition as described herein comprises the anti-CD19 antibody (e.g., obexelimab) in a predominately intact form. Various methods may be used to measure a composition, for example, through non-reduced CE-SDS. In some embodiments, a composition comprises ≥90% intact antibody (e.g., as measured by non-reduced CE-SDS). In some embodiments, a composition comprises ≥95% intact antibody (e.g., as measured by non-reduced CE-SDS).

### EXAMPLES

### Example 1. Conditions for Media Optimization and Feed Strategy of Obexelimab

This example outlines an exemplary feed strategy that can be used to produce anti-CD19 antibodies (e.g., obexelimab). A mammalian cell line was engineered via multiple rounds of transduction to stably co-express the heavy and light chains of obexelimab. The mammalian cells then underwent single cell cloning with a lead clone chosen to establish a cryogenically preserved master cell bank and a working cell bank that then served as the clonal production cell line. A vial of the clonal production cell line was thawed from the working cell bank and then underwent a convention seed culture expansion. An exemplary procedure for expanding the cells to a production process is outlined in Table 2.

TABLE 2
Upstream Process Flow Diagram

| Process Parameters | Process Steps |
|---|---|
| 36.0-38.0° C. | Working Cell Bank vial thaw |
| 120-150 seconds | |
| 36.0-38.0° C., 3 days | Cell expansion in 250 mL shake flask |
| 36.0-38.0° C., 2 days | N-7 cell expansion in 500 mL shake flask |
| 36.0-38.0° C., 2 days | N-6 cell expansion in 1 L shake flask |
| 36.0-38.0° C., 2 days | N-5 cell expansion in 3 L shake flask |
| 36.0-38.0° C., 2 days | N-4 cell expansion in 2 × 3 L shake flasks |
| 36.0-38.0° C., 2 days | N-3 cell expansion in 50 L WAVE |
| | bioreactor |
| 36.0-38.0° C., 2 days | N-2 cell expansion in 2 × 50 L WAVE |
| | bioreactors |
| pH, Dissolved O₂ (DO) | N-1 cell expansion in 200 L single-use |
| 36.0-38.0° C., 3 days | bioreactor |
| pH, DO, 36.0-38.0° C. | 2000 L production bioreactor |
| (before temperature shift); | |
| 33.0-35.0° C. | |
| (post temperature shift) | |
| 8-12 days | |

Following a conventional upstream expansion, cells were maintained at high viability in a 2000 L fed-batch production bioreactor. The duration of the production process may range from 8-12 days. As outlined in Table 3, an exemplary feeding schedule maintained cell viability greater than or equal to 90% (e.g., 95%, 96%, 97%, etc.) while ensuring good productivity. As required, alkali 7.5% sodium bicarbonate and antifoam (3% simethicone) were added to the bioreactor.

TABLE 3
Bioreactor (brx) Nutrient Feeding Schedule

| Timing | Feed |
|---|---|
| Base Medium | Thermo Fisher CD OptiCHO ™ AGT, |
| | Glutamine, Poloxmer188 |
| Pre-inoculation | 26.0 (25.5-26.5) ml/L, Cytiva Cell Boost4 ™ |
| Day 2 | 350 g/L of current brx volume of Thermo |
| | Fisher CD CHO 5x feed AGT ™ |
| | supplemented with glutamine and galactose |
| Day 2 | 20 g/L of current brx volume of asparagine |
| | and serine |
| Day 2 | 20 g/L of current brx volume of tyrosine and |
| | cystine |
| Between Day 6 and Day 11 | Thermo Fisher CD CHO 5x feed AGT ™ |
| at the first instance of | supplemented with galactose |
| culture glucose decreasing | |
| below 5.5 g/L | |
| Day 5 | Cystine |
| Between Days 7 and 11 at | 15.23 g/L of current brx volume glucose |
| least one day after the first | |
| instance of culture glucose | |
| decreasing below 4.5 g/L | |

Nutrient supplementation via fed-batch supported antibody production by allowing cell density to increase without sacrificing cell viability, thereby delaying culture viability crash. Cells were then harvested from the production phase once cell viability decreased to below 97%, typically between day 8 and day 12 of the fed-batch production process. Cell viability above 97% was maintained throughout the production process for this period of time, ensuring key product quality attributes were maintained such as minimizing low molecular weight species (<5%) and producing the desired N-glycan profile for an extended therapeutic half-life (sialyation >25% and mannose-5<1%).

### Example 2. Exemplary Conditions for High Cell Viability and Low Molecular Species Generation

This example outlines exemplary conditions for maintaining high viable cell density (VCD). Cell viability of greater than or equal to 97% was achieved by control of cell growth, the pH, dissolved oxygen (DO), pressure of carbon dioxide (pCO2), and temperature. The pH of the fed-batch bioreactor was controlled at a set point of 7.0 using sodium bicarbonate (NaHCO₃) and CO₂, wherein the DO was maintained at 40%.

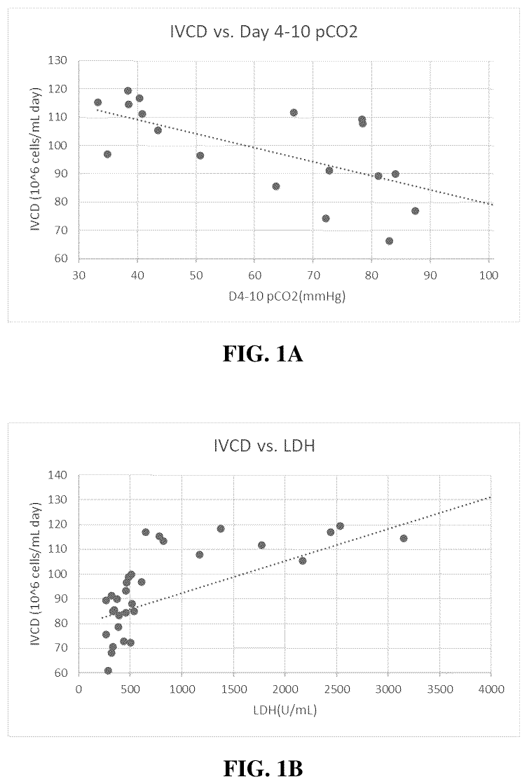

The pCO2 was continuously monitored over the course of the incubation period, including the cell expansion process and the production process. Controlling the pCO2 to be 80-130 mmHg between days 4-10 of the production process prevented cell overgrowth (see FIG. 1A). In this way nutrient exhaustion and premature cell culture crash was prevented, as evidenced by the concentration of lactate dehydrogenase (LDH), a marker for cell damage (see FIG. 1B).

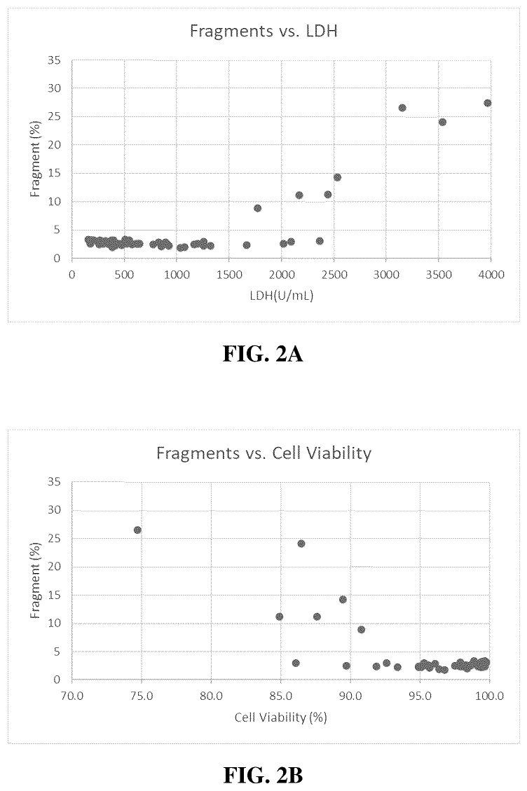

In maintaining these media conditions and utilizing the feeding strategy outlined in Table 3, key product quality attributes are produced in the harvested anti-CD19 antibody produced according to these methods. For example, as cell culture health decreased, the percentage of low molecular weight species in the harvested production process increased drastically (see FIG. 2A and FIG. 2B). Therefore, keeping cell viability high reduced the presence of these undesirable low molecular weight species and reduced the need for intensive downstream purification.

Initially, the target temperature was set to 37.0° C. to facilitate cell growth. Subsequently, the target temperature was shifted to 34.0° C. when the viable cell density (VCD) exceeded 5.0×10⁶ cells/mL. Shifting the temperature in this manner assisted in controlling the growth rate of the cells, preventing early crash and nutrient depletion, and in doing so maintained a high cell viability throughout the production process.

### Example 3. Overview of Downstream Processing

This example provides a general overview of an exemplary downstream purification, viral inactivation, and filtration steps for extracting the antibody product. Briefly, post clarification of the harvest was achieved by conventional means wherein the antibody product was captured by affinity chromatography on Protein A resin, followed by low pH viral inactivation. The eluate was subjected to further purification by anion exchange (AEX) chromatography and cation exchange (CEX) chromatography. After the CEX step, the eluate was filtered to remove virus particles and further filtered by ultrafiltration/diafiltration (UF/DF), wherein the final pool obtained was diluted to the target drug substance concentration of 125 g/L.

### Example 4. Characterization of Anti-CD19 Antibody Obexelimab

This example outlines exemplary procedures used and results obtained from characterizing an anti-CD19 antibody (e.g., obexelimab), including the glycosylation profile of an anti-CD19 antibody obexelimab and the desialylation assay for determining the charge profile therein.

### Characterization of Intact Antibody and Monomer

The composition of the antibody were characterized as comprising ≥90% intact antibody as determined by non-reducing capillary electrophoresis-sodium dodecyl sulfate (CE-SDS). Purity of the monomeric antibodies as determined by size exclusion-ultra performance liquid chromatography (SE-UPLC) was greater than or equal to 95%. Particularly, obexelimab produced in a method as described in examples 1-3 had ≥97% intact antibody as measured by CE-SDS and ≥98% monomer as measured by SE-UPLC.

### LC-MS/MS Identification of N-Glycosylation Sites

N-glycosylation sites in the antibody products were determined by conventional liquid-chromatography tandem mass spectrometry (LC-MS/MS) methodologies. Briefly, all samples were compared and analyzed by reduced peptide mapping using data from Lys-C and trypsin digestion with and without PNGase F treatment, an enzyme for removal of N-linked oligosaccharides from glycoproteins. This allows for distinguishing the glycosylated and deglycosylated species by retention time. LC-MS/MS of obexelimab identified N301 of the heavy chain as a N-glycosylation site.

### N-Glycan Profiling Using UPLC-FLD

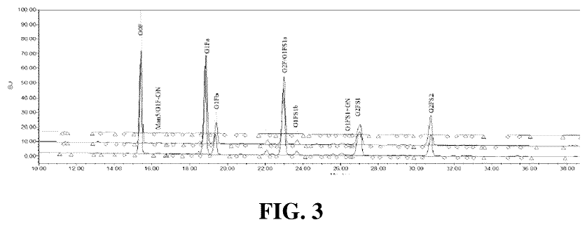

The N-glycan profile of the antibody products was characterized after glycan release, purification, 2-aminobenzamide labeling (to fluorescently label glycans), and Ultra Performance Liquid Chromatography-Fluorescent Light Detection (UPLC-FLD) analysis. The chromatogram of the N-glycan profile of the antibodies is visualized in FIG. 3, and the composition of each profile is summarized in Table 4. These results indicate a unique N-glycosylation composition of the obexelimab antibody product, notably that the presence of sialylated N-glycans for all samples is greater than 20%. Furthermore, as demonstrated by the varying harvest timings as noted in Table 4, maintaining high cell viability throughout the entire production process improved the percentage of highly sialyated N-glycans.

TABLE 4
N-Glycan Profiles of Antibody Products

| | Harvest Criteria of Production Process | | |
|---|---|---|---|
| | | ≤Day 12 or when | |
| | Cell viability ~80% | cell viability | ≤Day 12 |
| | or Day 15, | is ~90%, | while cell |
| | whichever occurred | whichever occurred | viability |
| | first | first. | is ≥97%. |
| N-Glycan Profile | Test Result (%) UPLC-FLD | | |
| G0F | 32.9 | 21.1 | 21.6 |
| Man5/G1FGN | 0.3 | 0.3 | 0.2 |
| G1Fa | 22.0 | 23.5 | 23.0 |
| G1Fb | 6.7 | 5.9 | 5.9 |
| G2F/G1FS1a | 14.5 | 20.3 | 20.1 |
| G1FS1b | 1.6 | 1.5 | 1.6 |
| G1FS1 + GN | 0.2 | 0.2 | 0.2 |
| G2FS1 | 6.7 | 11.5 | 9.5 |
| G2FS2 | 6.7 | 7.7 | 10.7 |
| Others | 8.5 | 8.0 | 7.2 |
| Group of | 21.9 | 28.6 | 32.7 |
| sialylated | | | |
| N-glycans | | | |

icIEF-Desialylation Assay for Charge Profiling

Imaged capillary isoelectric focusing (icIEF) is a technique known in the art for characterizing charge variants of proteins. Under an electric current, charge variants with differing isoelectric points are focused and separated out. In the case of obexelimab, the high presence of sialylated glycans (˜20%) obfuscates charge variants formed from the side chains of amino acids. To overcome this, the samples are pre-treated with Sialidase A, which removes sialic acid, thereby allowing the quantification of acidic, basic, and main peaks resolved by icIEF. Particularly, obexelimab produced in a method as described in examples 1-3 was characterized through icIEF-disalyation as having a main charge variant peak of approximately 50.3%, an acidic charge variant peak of approximately 27.6%, and a basic charge variant peak of approximately 22%.

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. A composition comprising
an anti-CD19 antibody comprising
a light chain variable region comprising a lCDR1 defined by SEQ ID NO: 1, a lCDR2 defined by SEQ ID NO: 2, and a lCDR3 defined by SEQ ID NO: 3; and
a heavy chain variable region comprising a hCDR1 defined by SEQ ID NO: 4, a hCDR2 defined by SEQ ID NO: 5, and a hCDR3 defined by SEQ ID NO: 6,
wherein the anti-CD19 antibody comprises an Fc region comprising S267E and L328F substitutions;
wherein the anti-CD19 antibody comprises an N-glycan profile comprising ≥20% sialylated glycans.

2. (canceled)

3. The composition of claim 1, wherein the N-glycan profile of the antibody comprises ≥25%, ≥26%, 28%, or 30% sialylated glycans.

4. The composition of claim 1, wherein the N-glycan profile of the antibody comprises ≤5%, 4%, 3%, 2% or 1% mannose-5 glycans.

5-8. (canceled)

9. The composition of claim 1, wherein the light chain variable region comprises SEQ ID NO: 7 and the heavy chain variable region comprises SEQ ID NO: 8.

10. The composition of claim 1, wherein the anti-CD19 antibody comprises a light chain comprising SEQ ID NO: 9 and a heavy chain comprising SEQ ID NO: 10.

11-13. (canceled)

14. The composition of claim 1, wherein the composition comprises ≤75 ng/mg host cell protein (HCP).

15-21. (canceled)

22. The composition of claim 1, wherein the composition comprises a main charge variant that constitutes ≥28% of charge variants by icIEF-Desialylation.

23. The composition of claim 1, wherein the composition comprises a main monomer peak that constitutes ≥95% of the total antibody measured by SE-UPLC Purity.

24. The composition of claim 1, wherein the composition comprises high molecular weight species that constitute ≤5.0% of the total antibody measured by SE-UPLC Purity.

25. The composition of claim 1, wherein the composition comprises low molecular weight species that constitute ≤5.0% of the total antibody measured by SE-UPLC Purity.

26. The composition of claim 1, wherein the composition comprises ≥90% intact antibody as measured by non-reduced CE-SDS.

27. A liquid composition comprising an anti-CD19 antibody at a concentration ranging from 113-138 mg/ml, wherein the anti-CD19 antibody comprises
a light chain variable region comprising a lCDR1 defined by SEQ ID NO: 1, a lCDR2 defined by SEQ ID NO: 2, and a lCDR3 defined by SEQ ID NO: 3; and
a heavy chain variable region comprising a hCDR1 defined by SEQ ID NO: 4, a hCDR2 defined by SEQ ID NO: 5, and a hCDR3 defined by SEQ ID NO: 6, and
wherein the anti-CD19 antibody comprises an Fc region comprising S267E and L328F substitutions;
wherein the composition comprises a main charge variant that constitutes ≥28% of charge variants by icIEF-Desialylation.

28-33. (canceled)

34. A method of manufacturing an anti-CD19 antibody, comprising
culturing mammalian cells engineered to express the anti-CD19 antibody in a fed-batch production process under conditions such that cell viability ≥95% is maintained during the fed-batch production process,
wherein the anti-CD19 antibody comprises a light chain variable region comprising a lCDR1 defined by SEQ ID NO: 1, a lCDR2 defined by SEQ ID NO: 2, and a lCDR3 defined by SEQ ID NO: 3, and a heavy chain variable region comprising a hCDR1 defined by SEQ ID NO: 4, a hCDR2 defined by SEQ ID NO: 5, and a hCDR3 defined by SEQ ID NO: 6, and
wherein the anti-CD19 antibody comprises an Fc region comprising S267E and L328F substitutions.

35. (canceled)

36. The method of claim 34, wherein the conditions comprise one or more of the following steps:
feeding cells with glutamine at day 2 of the fed-batch production process;
feeding cells with galactose at day 2 of the fed-batch production process;
feeding cells with asparagine at day 2 of the fed-batch production process;
feeding cells with serine at day 2 of the fed-batch production process;
feeding cells with tyrosine at day 2 of the fed-batch production process;
feeding cells with cystine at day 2 of the fed-batch production process;
feeding cells with galactose between day 6 and day 11 of the fed-batch production process;
feeding cells with cystine at day 5 of the fed-batch production process; and
providing glucose between day 6 and day 11 of the fed-batch production process.

37-39. (canceled)

40. The method of claim 34, wherein the conditions comprise a step of controlling pCO2 between 80 to 130 mmHg during the fed batch production process.

41-59. (canceled)
